(19) United States
(12) Patent Application Publication
(10) Pub. No.: US 2004/0210181 A1
(43) Pub. Date: Oct. 21, 2004

(54) Drainage implant for draining aqueous humour from the anterior aqueous chamber of the eye into schlemm's canal

(76) Inventors: Clemens Vass, Vienna (AT); Winfried Mayr, Moedling (AT)

Correspondence Address: HELLER EHRMAN WHITE & MCAULIFFE LLP, 1666 K STREET,NW, SUITE 300, WASHINGTON, DC, 20006, US

(21) Appl. No.: 10/476,160

(22) PCT Filed: Apr. 26, 2002
(86) PCT No.: PCT/AT02/00130

(30) Foreign Application Priority Data
Apr. 26, 2001 (AT) A 679/2001

(51) Int. Cl. A61M 5/00; A61M 25/00

(52) U.S. Cl. 604/8; 604/284

(57) ABSTRACT

The invention relates to an implant for draining the aqueous humour from the anterior aqueous chamber of the eye into Schlemm's canal. Said implant comprises a tubular proximal part (1) with at least one lumen, whose open end (2, 3) can be introduced into the anterior aqueous chamber for draining the aqueous humour and a tubular distal part (5) that is open at both ends (6) and that has at least one lumen connected to the proximal part (1) to form a substantially T-shaped implant, whereby said distal part (5) can be introduced into Schlemm's canal. To produce an implant of this type, which can be securely fixed, at least one plate-shaped fixing element (7) is provided, said element stabilising the implant on the sclera. The fixing element is connected by the central section to the distal part (5) on the opposite side of the latter to the proximal part (1) and comprises at least one eyelet (8) or similar for fixing with a suture.

The present invention relates to an implant for draining aqueous humor from the anterior aqueous chamber of the eye into Schlemm's canal, with a tubular proximal part with at least one lumen, which can be introduced with its open end into the anterior aqueous chamber of the eye for draining the aqueous humor, and a tubular distal part, which is open at both ends, with at least one lumen, which is connected with the proximal part for forming an essentially T-shaped implant, which distal part can be introduced into Schlemm's canal.

The implant described is used primarily in the case of glaucoma, the so-called green star. This disease is characterized by a chronically progressing lesion of the optic nerve with the main risk factor of an increased intra-ocular pressure. Approximately 2 μL of aqueous humor are produced per minute in the interior of the eye and drain through the trabecular network, located in the angle of the iris of the anterior aqueous chamber of the eye, into Schlemm's canal and from there, over the collecting tubules and the aqueous humor veins into the venous system. The main physiological resistance to this aqueous humor drainage lies in the juxtacanalicular part of the trabecular network, that is, between the anterior aqueous chamber of the eye and Schlemm's canal. In the case of chronic open angle glaucoma, it is this resistance, which is increased pathologically.

At the present time, fistulating glaucoma operations (trabeculectomy) represent the standard method for the surgical lowering of the intra-ocular pressure of glaucoma patients. For this procedure, the aqueous humor is drained through a scleral wound from the anterior aqueous chamber of the eye under the conjunctiva, where the aqueous humor is taken up by veins and transported away. The short-term successes at a rate of about 90% are acceptable. However, in the course of a few years the healing of the wound not infrequently leads to an occlusion of the fistula and, with that, to a late failure of the surgery.

New surgical techniques of non-penetrating glaucoma surgery (deep sclerectomy, viscocanalostomy) have recently demonstrated that, in the case of chronic open angle glaucoma, Schlemm's canal can be prepared reproducibly and, moreover, also be used functionally at least in the case of viscocanalostomy.

The EP 0 898 947 A2 disclosed an implant, which is implanted in Schlemm's canal within the scope of a viscocanalostomy, for a permanent expansion. In the case of deep sclerectomy, fistulation under the conjunctiva is aimed for, an attempt being made partially to support this by the use of implants. Nevertheless, this method was also unable to solve the problem of post-operative scarring. For this reason, the medium term success rate is similar to that of trabeculectomy. It is a common feature of all non-penetrating methods of glaucoma surgery that a thin layer of tissue, also referred to as a trabecular-descemetic window, is retained and exerts an effect, which cannot be defined precisely, on the drainage resistance.

It is also the objective of the device, which is described in the WO 00/13627, to improve the drainage of aqueous humor from the anterior aqueous chamber into Schlemm's canal and, at the same time, to keep the latter open. For this purpose, a stent, which expands the trabecular network and has openings directed towards the latter, is implanted in the Schlemm's canal.

In complicated cases, perhaps also after several prior surgical procedures, drainage implants have been used for some time (Molteno, British Journal of Ophthalmology, 1977, 61:120-125; Krupin, Ophthalmology 1988, 95:1174-1180; Schocket, Ophthalmology 1982, 89:1188-1194); Smith, Ophthalmology 1993, 100:914-918; Coleman, American Journal of Ophthalmology 1995, 120:23-31), all of which are constructed on the same basic principle; a thin, small tube (mostly of silicone), which is introduced with an open end into the anterior aqueous chamber of the eye, drains the water to a plate or cerclage band, which is fixed far behind at the eyeball. Consequently, a capsule is formed about this plate or cerclage band, the flow resistance (and therefore the intra-ocular pressure) being determined by the permeability of this capsule, as well as by the surface of the capsule. This method also suffers from the problem of scarring.

Spiegel (Ophthalmic Surg Lasers 1999; 30:492-494) described a method for drainage from the anterior aqueous chamber of cadaver eyes directly into Schlemm's canal. For this purpose, he used a small silicone tube with an external diameter of 0.15 mm and an internal diameter of 0.05 mm.

In the international patent application WO 00/64393 A1, an implant of the objective type is described for drainage of the aqueous humor from the anterior aqueous humor of the eye into Schlemm's canal. This can be introduced, on the one hand, with its open, proximal part into the anterior aqueous chamber of the eye and, on the other, with the distal parts on either side into Schlemm's canal.

In Spiegel's work, as well as in the above-mentioned patent application WO 00/64393 A1, the problem of fixing the drainage implant stably remains unsolved.

It is an object of the present invention to create an implant for draining aqueous humor from the anterior aqueous chamber of the eye directly into Schlemm's canal and, above all, to enable the drainage implant to be fixed stably. Moreover, the present implant, as far as possible is not to be subjected to any rejection reactions, such as an encapsulation.

Provisions are therefore made pursuant to the invention that, for stabilizing the implant at the sclera, at least one plate-shaped fixation element is provided, which is connected with the distal part in the central region and at the side of the distal part, which is averted from the proximal part. A rapid and simple stabilization and fixation of the implant by means of sutures is thus made possible by the inventive, plate-shaped fixation element. Moreover, the function of the present implant, in its implanted position is not affected by encapsulation, since the proximal end of the implant protrudes into the tissue-free, anterior aqueous chamber of the eye and the distal part of the implant is localized in Schlemm's canal. Because of the absence of connective tissue in these regions, there are also no connective tissue-related foreign body reactions. The proximal part of the implant is pushed through a surgically produced canal into the anterior aqueous chamber of the eye so that, over the open front end, the aqueous humor can be drained over the proximal part and the distal part. If the at least one plate-shaped fixation element is sufficiently thin, the fixation can be accomplished by piercing this element with the help of a surgical needle.

In addition, at least one eyelet or the like can be provided for the suture fixation in the palate-shaped fixation element. Such an opening also offers advantages, since sclera can grow through it.

Instead of or in addition to such eyelets, puncture sites of reduced thickness can also be provided at plate-shaped fixation element. These puncture sites can be punctured more easily with the help of a surgical needle.

Advantageously, the proximal part has two lumina, one lumen in each case ending in one end of the distal part. A lumen of the proximal part thus forms an L-shape with the lumen of the distal part. The two-lumen or multi-lumen construction has the advantage that, if one lumen becomes blocked, the implant remains intact at least partially.

In order to be able to introduce these open ends of the distal part of the implant atraumatically on both sides into the Schlemm's canal, they are rounded off in accordance with a further distinguished feature of the invention.

To facilitate the introduction of the proximal part into the front aqueous chamber and also to facilitate the absorption of aqueous humor over the open end of the proximal part, the open end of the proximal part can be constructed beveled.

In order to use not only those collecting tubules for draining the aqueous humor from Schlemm's canal into the veins, which start out just behind the distal end of the implant at the outer wall of Schlemm's canal, but also those collecting tubules, which start out along the part implanted in Schlemm's canal, the distal part of the implant, at least at the regions adjoining the two open ends, may have cross connections from the at least one lumen of the distal part to the outside. These cross connections between the lumen of the distal part and its outside can be constructed in different ways, for example, by appropriate circular or oval openings or boreholes or the like.

One embodiment of such cross connections is formed by tubular sections, which are halved in the longitudinal direction, with formation of lateral openings between circular tubular segments.

The internal diameter of the lumina in the proximal and distal part of the implant is between 0.02 and 0.2 mm and preferably between 0.03 and 0.1 mm. These dimensions ensure adequate flow of aqueous humor of the anterior aqueous chamber of the eye into Schlemm's canal.

The preferred external diameter of the proximal part of the implant is between 0.1 and 0.6 mm.

The preferred external diameter of the distal part of the implant is between 0.05 and 0.3 mm. In this connection, the lower limit is determined by the manufacturing process and the upper limit by the dimensions of Schlemm's canal.

The implantation is facilitated owing to the fact that the distal part of the implant, corresponding to Schlemm's canal, is constructed curved in the direction of the proximal part.

Preferably, at least parts of the implant are manufactured from plastic, preferably from a silicone or Teflon. These materials offer preferred processing possibilities as well as an optimum elasticity, which offers advantages during the implantation.

Likewise or additionally, it is possible that at least parts of the implant consist of metal, stainless steel, titanium, silver, gold or platinum being particularly suitable.

In order to deliberately cause desirable biological reactions or avoid undesirable biological reactions, at least parts of the implant may be coated or at least parts of the surface may be finished. For example, Teflon parts may be roughened with an electron beam, in order to an enable the implant to grow in better.

The invention is described below, without limiting the general inventive concept, by means of examples and with reference to the drawings, in which

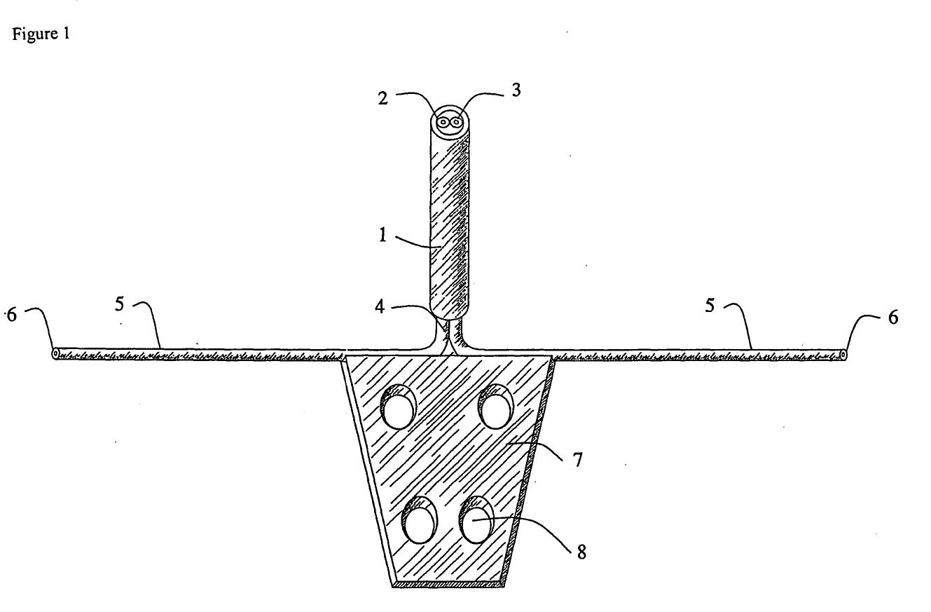

FIG. 1 shows a diagrammatic representation of a drainage implant,

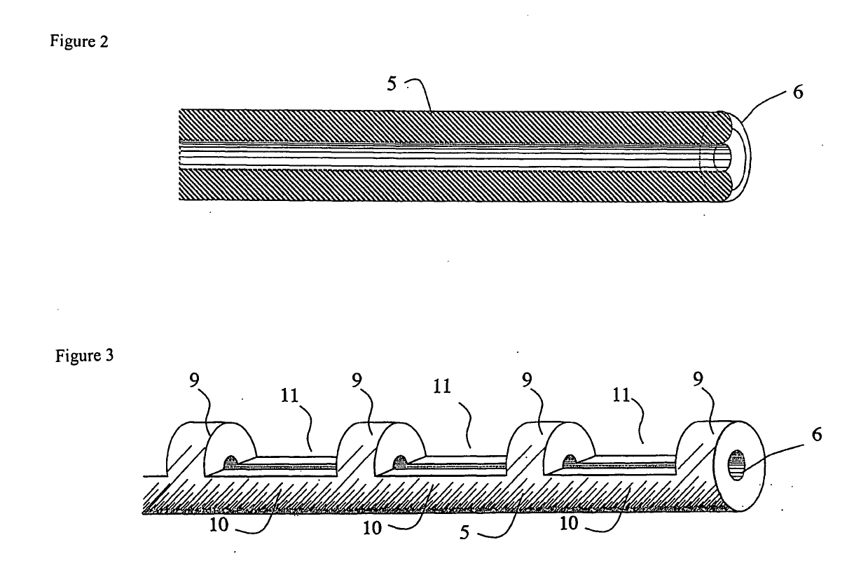

FIG. 2 shows a diagrammatic representation of a section of a small tube of the distal part of an implant with rounded ends in a sectional representation,

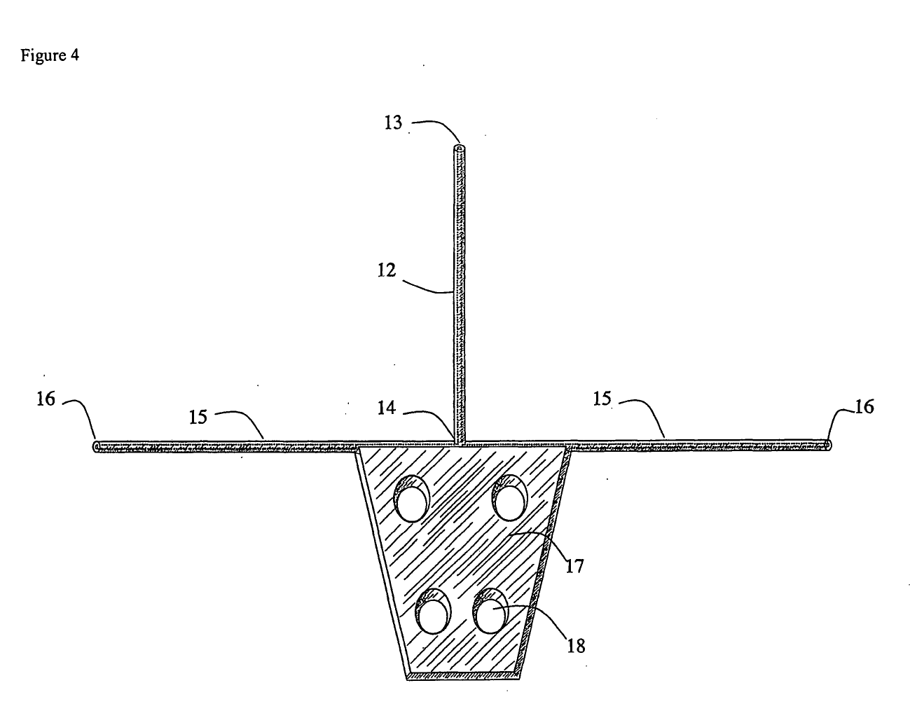

FIG. 3 shows a diagrammatic representation of a further example of a section of a small tube of the distal part and

FIG. 4 shows a diagrammatic representation of a further example of a drainage implant.

FIG. 1 shows an example of an implant for draining the aqueous humor from the anterior aqueous chamber of the eye into Schlemm's canal. The implant comprises a tubular proximal part 1, which has two separate, parallel lumina and can be introduced with its open end 2, 3 into the anterior aqueous chamber of the eye, for draining the aqueous humor and a tubular distal part 5, which is open at both ends 6 and formed by the branching of the two lumina, which is provided at the distal end 4 of the proximal part 1 in opposite directions and approximately at right angles, with the formation of two diametrically extending, separate small tubes 5. A plate-shaped fixation element is mounted opposite the right angled branching 4 of the two small tubes, in order to enable the implants to be stabilized and fixed by means of sutures. The plate-shaped fixation element 7 may contain one or more eyelets 8 or the like, by way of which appropriate sutures can be produced. Instead of or in addition to such eyelets 8, puncture sites of reduced thickness, which can be punctured with a surgical needle, may also be provided, in order to bring about appropriate fixation of the implant. In comparison to an implant with a single lumen, one with two or more lumina has the advantage that, if one lumen is blocked, the second lumen remains intact so that, even if only limited, drainage of the aqueous humor remains possible. The lumina can be formed by appropriate dummies during the manufacture of the implant or subsequently by introducing appropriate tubes or the like in the proximal and distal parts 1, 5.

The open ends 6 of the distal part can be rounded off, so that they can be introduced atraumatically on both sides into Schlemm's canal (FIG. 2).

In FIG. 3, one end 6 of the distal part 5 of the implant is shown as a further example. It is constructed so that several circular tubular segments 9 are connected to one another by small tubes 10, which are halved in the longitudinal direction. The circular, tubular segments 9 support the Schlemm's canal, and the lateral openings 11, formed between the circular, tubular segments 9, point to the outer wall of the Schlemm's canal, where the collecting tubules of the eye drain the aqueous humor to the aqueous humor veins. It is also possible to equip only the region of the distal part 5, adjoining the open end 6 of the distal part 5, with such openings 11 in order to provide sufficient flow resistance in the upstream parts of the small tubes.

FIG. 4 shows a further example of an implant, which comprises a single lumen proximal part 12, which is open at its proximal end 13 and can be introduced into the anterior aqueous chamber of the eye. Furthermore, the implant comprises a tubular, distal part 15, which is open at both ends 16 and connected at its central region approximately at right angles with the other end of the proximal part 12, lying opposite to the proximal end 13. Opposite to the right-angled branching 14 of the two parts 12, 15, a plate-shaped fixation element 17, which may contain one or more eyelets 18, is mounted, in order to enable the implant to be stabilized and fixed with sutures.

For the surgery, Schlemm's canal is exposed by viscocanalostomy in that, initially, a superficial scleral flap, approximately ⅓ the thickness of the sclera, and subsequently a deep scleral flap, approximately 80% of the thickness of the sclera are prepared. The deep scleral flap severed at the front edge of Schlemm's canal. As a result an intrascleral cavity is formed, which can accommodate the plate-shaped fixation element 7; 17 of the implant. The two incision openings of Schlemm's canal are probed with a thin cannula and Schlemm's canal is dilated somewhat by injecting a viscoelastic fluid. The plate-shaped fixation element 7, 17 is placed in the bed of the deep scleral flap. The anterior aqueous chamber of the eye is opened anterior of Schlemm's canal by an inclined stab incision of suitable diameter and the two small tubes in the proximal section 1, which is connected in parallel, are cut off at a suitable length at such an angle, that the opening points to the front and are then introduced through the stab incision into the anterior aqueous chamber of the eye. The distal ends 6 of the two small tubes are thereupon introduced into Schlemm's canal.

Utilizing the eyelets 8; 18, the plate-shaped fixation element 7, 17 is sewn with a suitable suture material to the sclera and the superficial scleral flap is subsequently closed off tightly over the device.

The plate-shaped fixation element 7, 17 can be connected with the distal part 5 of the implant by gluing or also produced in one piece with the latter, for example by injection molding. In particular, plastics such a silicone or Teflon are used as materials for at least parts of the implant. However, metals such as platinum or stainless steel can also be used. Compared to metals, plastics have the advantage of an increased elasticity, so that the implantation, especially the introduction of the open ends of the distal part of the implant, is facilitated. The inventive fixation plate 7, 17 may be a centrally disposed plate-shaped fixation element or several fixation elements of different shapes.

1. Implant for draining aqueous humor from the anterior aqueous chamber of the eye into Schlemm's canal, with a tubular proximal part (1, 12) with at least one lumen, which can be introduced with its open end (2, 3, 13) into the anterior aqueous chamber of the eye for draining the aqueous humor, and a tubular distal part (5, 15), which is open at both ends (6, 16) with at least one lumen, which is connected with the proximal part (1, 12) for forming an essentially T-shaped implant, which distal part (5, 15) can be introduced into Schlemm's canal, characterized in that, for stabilizing the implant at the sclera, at least one plate-shaped fixation element (7, 17) is provided, which is connected in the central region and at the side of the distal part (5, 15), averted from the proximal part (1, 12), with the distal part (5, 15).

2. The implant of claim 1, characterized in that the plate-shaped fixation element (7, 17) has at least one eyelet (8, 18) or the like for fixing the suture in position.

3. The implant of claims 1 or 2, characterized in that the plate-shaped fixation element (7, 17) has at least one puncture site of reduced thickness for fixing the suture in position.

4. The implant of one of the claims 1 to 3, characterized in that the proximal part (1) has two lumina, one of which emerges at either end (6, 16) of the distal part (5, 15).

5. The implant of one of the claims 1 to 4, characterized in that the two open end (6, 16) of the distal part (5, 15) are rounded off.

6. The implant of one of the claims 1 to 5, characterized in that the open end (2, 3, 13) of the proximal part (1, 12) is beveled.

7. The implant of one of the claims 1 to 6, characterized in that the distal part (5, 15) has cross connections from the at least one lumen of the distal part (5, 15) to the outside at least at the regions adjoining the two open end (6, 16).

8. The implant of claim 7, characterized in that the cross connections are formed by sections (10) of small tubes, which are halved in the longitudinal direction with formation of lateral openings (11) between circular segments (9) of the small tubes.

9. The implant of one of the claims 1 to 8, characterized in that the internal diameter of the lumina (2, 3, 5, 15), forming the proximal part (1, 12) and the distal part (5, 15), is between 0.02 and 0.2 mm and preferable between 0.03 and 0.1 mm.

10. The implant of one of the claims 1 to 9, characterized in that the external diameter of the external part (1, 12) is between 0.1 mm and 0.6 mm.

11. The implant of one of the claims 1 to 10, characterized in that the external diameter of the distal part (5, 15) is between 0.05 and 0.3 mm.

12. The implant of one of the claims 1 to 11, characterized in that the distal part (5, 15) is curved in the direction of the proximal part (1, 12)

13. The implant of one of the claims 1 to 12, characterized in that at least parts of the implant consist of plastic, preferably of silicone or Teflon.

14. The implant of one of the claims 1 to 13, characterized in that at least parts of the implant consist of metal, preferably of stainless steel, titanium, silver, gold, or platinum.

15. The implant of one of the claims 1 to 14, characterized in that at least parts of the implant are coated.

16. The implant of one of the claims 1 to 15, characterized in that at least parts of the surface are finished, for exampled, by roughening.
